(19) United States
(12) Patent Application Publication
(10) Pub. No.: US 2006/0122651 A1
(43) Pub. Date: Jun. 8, 2006

(54) Use of mechanical restitution to predict hemodynamic response to a rapid ventricular rhythm

(76) Inventor: Teresa A. Whitman, Dayton, MN (US)

Correspondence Address: MEDTRONIC, INC., 710 MEDTRONIC PARK, MINNEAPOLIS, MN, 55432-9924, US

(21) Appl. No.: 11/004,251

(22) Filed: Dec. 3, 2004

(51) Int. Cl. A61N 1/36

(52) U.S. Cl. 607/14

(57) ABSTRACT

An implantable cardiac stimulation device and associated method for predicting the hemodynamic response to a rapid heart rhythm. The system includes an implantable cardiac stimulation device and associated sensors of electrical and mechanical heart function. The associated method includes measuring a mechanical restitution (MR) parameter or surrogate thereof, performing a comparative analysis of the MR parameter, and predicting an unstable or stable hemodynamic response to a rapid heart rate based on the comparative analysis. If an unstable hemodynamic response to a rapid rhythm is predicted, a more aggressive menu of arrhythmia therapies may be programmed to treat tachycardia. If a stable hemodynamic response is predicted, a less aggressive menu of therapies may be programmed to treat tachycardia.

### FIELD OF THE INVENTION

The present invention relates generally to cardiac monitoring and electrical stimulation devices and more particularly to an implantable system for monitoring mechanical restitution to predict hemodynamic tolerance of a rapid rhythm.

### BACKGROUND OF THE INVENTION

Implantable cardioverter defibrillators (ICDs) generally detect tachycardia and fibrillation based on time intervals between cardiac events, i.e., P—P intervals in the atria and R—R intervals in the ventricles, derived from a cardiac electrogram (EGM) signal. In addition to this rate-based information, patterns of cardiac events, such as P—R intervals and R—P intervals, and EGM signal morphology may be used in discriminating between different types of arrhythmias. When an arrhythmia is detected and classified according to event intervals, interval patterns, morphology or other EGM information, an arrhythmia therapy is selected and delivered to terminate the arrhythmia with the desired result of restoring normal sinus rhythm.

Typically, ventricular tachycardia (VT) is detected based on a predetermined number of R—R intervals measured on a ventricular EGM signal falling within a VT detection zone. The ventricular tachycardia detection zone may be divided into a slow VT zone and a fast VT zone. A detected VT may then be treated with a menu of tiered therapies beginning first with less aggressive arrhythmia therapies and proceeding to more aggressive therapies if the VT persists or is accelerated. A less aggressive therapy may be anti-tachycardia pacing which requires less energy and is not painful to the patient compared to a more aggressive cardioversion shock. More serious arrhythmias, such as ventricular fibrillation (VF), are generally treated quickly with a cardioversion or defibrillation shock in order to quickly terminate the arrhythmia.

Rate or interval-based arrhythmia detection methods are limited in discriminating hemodynamically stable from unstable forms of VT. Analysis of interval patterns or morphology may aid in discriminating between supra-ventricular tachycardia and VT but does not provide information regarding the hemodynamic stability of a detected VT. Therefore, the hemodynamic status of the patient during a detected VT, is generally not taken into account when delivering a tachycardia therapy. Methods for discriminating between hemodynamically stable and unstable VT using hemodynamic or other physiological sensed parameters have been proposed. See for example, U.S. Pat. No. 5,176,137 issued to Erickson et al., U.S. Pat. No. 5,496,361 issued to Moberg et al., U.S. Pat. No. 6,477,406 issued to Turcott, U.S. Pat. No. 5,311,874 issued to Baumann et al., and U.S. Pat. No. 4,967,749 issued to Cohen.

### BRIEF DESCRIPTION OF THE DRAWINGS

Aspects and features of the present invention will be readily appreciated as the same becomes better understood by reference to the following detailed description when considered in connection with the accompanying drawings, in which like reference numerals designate like parts throughout the figures thereof and wherein:

FIG. 1 depicts an implantable medical device in which the present invention may be implemented;

FIG. 2 is a functional schematic diagram of the device shown in FIG. 1;

FIG. 3 is a flow chart summarizing steps included in a general method for practicing the present invention;

FIG. 4 is a flow chart summarizing steps included in a method for monitoring mechanical restitution to predict hemodynamic stability during a rapid heart rhythm;

FIG. 5 is a time line depicting the application of extra systolic pulses and the mechanical response to the extra systoles, which may be measured for estimating a slope of the MR curve;

FIG. 6 is an illustration of mechanical restitution curves representing a normal, healthy hemodynamic response and an abnormal, blunted hemodynamic response to extra systoles occurring over a range of ESIs;

FIG. 7 is a flow chart summarizing steps included in a method for predicting the hemodynamic response to a fast rhythm according to the present invention;

FIG. 8 is a flow chart providing details of a method for predicting the hemodynamic response to a fast rhythm by using a post-extra systolic mechanical function measurement according to the present invention;

FIG. 9 is a graph depicting ventricular pulse pressure plotted against diastolic interval;

FIG. 10A is a depiction of an EGM signal, a pressure signal, and an accelerometer signal illustrating methods for measuring the DI according to the present invention;

FIG. 10B is a depiction of a dP/dt signal illustrating a method for measuring the DI according to the present invention;

FIG. 11 is a flow chart of a method for predicting the hemodynamic response to a fast rate based on measurements of the DI and associated mechanical function according to the present invention;

FIG. 12 is a flow chart illustrating the use of measurements of DI differences to determine a MR parameter in predicting the hemodynamic response to a fast rate according to the present invention; and

FIG. 13 is a flow chart of a method for using MR parameter or surrogate measurements for discriminating stable from unstable VT during detection of a VT episode according to the present invention.

### DETAILED DESCRIPTION OF THE PREFERRED EMBODIMENTS

The present invention provides an implantable cardiac stimulation device and associated method for predicting the hemodynamic response to a rapid heart rhythm. Knowledge of the predicted hemodynamic response to a rapid rhythm is useful in selecting arrhythmia therapies, particularly tachycardia therapies. An unstable or stable hemodynamic response is predicted based on a measurement of a mechanical restitution parameter or surrogate thereof. The hemodynamic response to rapid ventricular rhythms will depend on the heart's mechanical restitution properties as well as the cardiac cycle length just prior to the first rapid heart beats. By measuring mechanical restitution, a prediction can be made as to whether a rapid rhythm like VT will be compensated for hemodynamically or is expected to result in hemodynamic insufficiency. If an unstable hemodynamic response to a rapid rhythm is predicted, a more aggressive menu of arrhythmia therapies may be programmed to treat tachycardia. If a stable hemodynamic response is predicted, a less aggressive menu of therapies may be programmed to treat tachycardia.

The system includes an implantable cardiac stimulation device and associated electrodes for sensing cardiac signals and delivering electrical stimulation pulses. The system further includes a mechanical sensor of heart function. The stimulation device includes sensing circuitry for receiving and processing sensed electrical and mechanical signals; pulse generating circuitry for delivering pacing pulses and high-voltage shocking pulses; timing and control circuitry for controlling the timing and delivery of electrical pulses to the heart; and a control system which may be in the form of a microprocessor. The microprocessor executes software programs stored in associated memory for controlling device functions including a method for measuring a mechanical restitution (MR) parameter or indicator thereof using the sensed mechanical and/or electrical signals and making a prediction of the hemodynamic response to a fast rhythm based on the MR parameter or indicator thereof.

In one embodiment, a MR parameter is determined as a slope of a MR curve. Mechanical function is measured from the mechanical sensor signal on extra-systolic beats, which may occur intrinsically or may be injected by the cardiac stimulation device at predetermined extra-systolic intervals (ESIs). A slope of the MR curve is calculated from two or more points determined by the mechanical response measured at two or more different ESIs. A relatively low slope predicts an unstable hemodynamic response to a rapid rhythm and a relatively steeper slope predicts a stable hemodynamic response to a rapid rhythm.

In another embodiment, mechanical function measurements are measured on a primary (S1) beat, on an extra systolic (S2) beat and a post-extra systolic (S3) beat. An enhanced mechanical response on the S3 beat evidences a normal mechanical restitution and predicts a stable hemodynamic response to a rapid rhythm. As such, the ratio of the S1/S3 or S2/S3 mechanical function measurements may be used for predicting the hemodynamic response to a rapid rhythm. A relatively high S1/S3 or S2/S3 ratio predicts an unstable hemodynamic response. A relatively low S1/S3 or S2/S3 ratio predicts a stable hemodynamic response.

In another embodiment, a parameter for use in predicting the hemodynamic response to a rapid rhythm may be determined. A parameter may be measured as a slope of a mechanical function versus diastolic interval (DI) curve. The DI is measured following an extra systole or at the onset of a rapid paced or intrinsic rhythm. The associated mechanical function is measured from the mechanical sensor signal. A slope is calculated from two or more points defined by two or more DIs and the associated mechanical function measurements. A low slope indicates the patient will be unable to hemodynamically compensate for a rapid rhythm.

An alternative surrogate parameter may be determined as the successive differences between DI measurements. If the DI does not increase during the first few beats of a rapid paced or intrinsic rate or in response to injected extra systoles, the patient will be unable to hemodynamically compensate for a rapid rhythm.

The prediction of a stable or unstable hemodynamic response to a rapid rhythm occurring in the future may be made based on periodic measurements of the MR parameter or surrogate thereof. The prediction may be used by a clinician in selecting arrhythmia therapies or by the implanted device to automatically select an arrhythmia therapy menu. Alternatively, a prediction may be made at the time of or just prior to arrhythmia detection. The prediction is based on a MR parameter or surrogate thereof measured during the first several rapid heartbeats. This prediction may be used by the implanted device to automatically select the arrhythmia therapy to be delivered in response to the subsequently detected arrhythmia.

The present invention is directed toward providing a system and method for monitoring mechanical restitution for use in predicting the hemodynamic response to a rapid rhythm. In a patient capable of compensating hemodynamically to a rapid heart rate, a less aggressive approach to treating a detected tachycardia may be appropriate. On the other hand, in a patient that is not able to compensate hemodynamically during an accelerated rhythm, a more aggressive therapeutic approach is desirable in order to prevent serious consequences of a hemodynamically unstable rhythm. Thus a method for predicting a patient's hemodynamic response to a rapid rhythm would be valuable in selecting arrhythmia therapies. Furthermore, it is desirable to know, either in advance or within the first several beats of a rapid rhythm, what the hemodynamic response is going to be with relative certainty. Hemodynamic measures, such as pulse pressure, may have considerable overlap between stable and unstable responders during the first several seconds of a rapid rhythm. Waiting for discriminatory hemodynamic evidence of a stable or unstable rhythm may therefore delay the selection and delivery of therapy.

Mechanical restitution refers to the mechanical response of a heart chamber to a premature systole and is thought to be related to the calcium handling properties of the cardiac myocytes. Abnormal calcium handling associated with heart failure results in altered mechanical restitution and manifests in impaired hemodynamic output. Mechanical restitution has been proposed as a parameter useful in monitoring the state of heart failure in U.S. Pat. No. 6,438,408, issued to Mulligan et al., hereby incorporated herein by reference in its entirety. Mechanical or hemodynamic measures of heart function, such as pulse pressure, wall motion or acceleration, may be measured at varying extra-systolic intervals to obtain a mechanical restitution curve represented by the mechanical measure of heart function versus extra-systolic interval (ESI). Various parameters characterizing the restitution curve may then be determined, such as a maximum slope of the steepest portion of the curve, the time constant, or the maximal response on the curve (the plateau), and used in assessing heart function.

The mechanical response to the first few rapid heart beats at the onset of an accelerated rhythm will depend on the ability of the myocardium to cycle calcium in and out of the extracellular space. This ability is reflected in the mechanical restitution properties of the heart. Knowledge of the myocardium's response to an extra systole can therefore aid in the prediction of the hemodynamic response to a rapid rhythm. Thus, a system and method are disclosed herein for measuring a mechanical restitution parameter or surrogate thereof for use in predicting or discriminating between hemodynamically stable and unstable tachycardia and in selecting arrhythmia therapies. The present invention may be embodied in an implantable medical device such as an ICD capable of delivering arrhythmia therapies and equipped with a sensor of mechanical heart function.

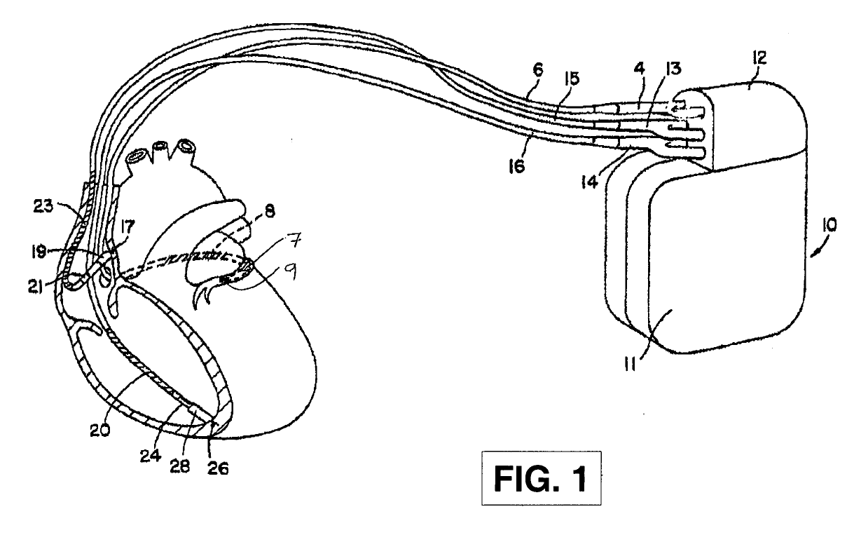

FIG. 1 depicts an implantable medical device in which the present invention may be implemented. Implantable medical device 10 is embodied as a multi-chamber pacemaker cardioverter defibrillator and is coupled to a patient's heart by three cardiac leads 6, 15, and 16. Device 10, also referred to herein as “ICD,” is capable of receiving and processing cardiac electrical signals and delivering electrical stimulation therapies to the heart, including cardiac pacing, cardioversion and defibrillation. Device 10 includes a connector block 12 for receiving the proximal end of a right ventricular lead 16, a right atrial lead 15 and a coronary sinus lead 6, used for positioning electrodes for sensing and stimulating in three or four heart chambers.

In FIG. 1, the right ventricular lead 16 is positioned such that its distal end is in the right ventricle for sensing right ventricular cardiac signals and delivering electrical stimulation therapies in the right ventricle. For these purposes, right ventricular lead 16 is equipped with a ring electrode 24, a tip electrode 26 optionally mounted retractably within an electrode head 28, and a coil electrode 20, each of which are connected to an insulated conductor within the body of lead 16. The proximal end of the insulated conductors are coupled to corresponding connectors carried by connector 14 at the proximal end of lead 16 adapted for electrical connection to device 10 via connector block 12.

The right atrial lead 15 is positioned such that its distal end is in the vicinity of the right atrium and the superior vena cava. Lead 15 is equipped with a ring electrode 21, a tip electrode 17 optionally mounted retractably within electrode head 19, and a coil electrode 23 for providing sensing and electrical stimulation therapies in the right atrium. The ring electrode 21, the tip electrode 17 and the coil electrode 23 are each connected to an insulated conductor with the body of the right atrial lead 15. Each insulated conductor is coupled at its proximal end to connector 13.

The coronary sinus lead 6 is advanced within the vasculature of the left side of the heart via the coronary sinus and great cardiac vein. The coronary sinus lead 6 is shown in the embodiment of FIG. 1 as having a defibrillation coil electrode 8 that may be used in combination with either RV coil electrode 20 or SVC coil electrode 23 for delivering electrical shocks for cardioversion and defibrillation therapies. Coronary sinus lead 6 is also equipped with a distal tip electrode 9 and ring electrode 7 for sensing electrical activity and delivering electrical stimulation therapies in the left ventricle of the heart. The coil electrode 8, tip electrode 9 and ring electrode 7 are each coupled to insulated conductors within the body of lead 6, which provide connection to the proximal connector 4. In alternative embodiments, lead 6 may additionally include ring electrodes positioned for left atrial sensing and stimulation functions.

The electrodes 17 and 21, 24 and 26, and 7 and 9 may be used in sensing and stimulation as bipolar pairs, commonly referred to as a “tip-to-ring” configuration, or individually in a unipolar configuration with the device housing 11 serving as the indifferent electrode, commonly referred to as the “can” or “case” electrode. Device 10 is preferably capable of delivering high-voltage cardioversion and defibrillation therapies in addition to anti-arrhythmia pacing therapies or other less aggressive electrical stimulation therapies for preventing or terminating an arrhythmia. Device housing 11 may serve as a subcutaneous defibrillation electrode in combination with one or more of the defibrillation coil electrodes 8, 20 or 23 for defibrillation of the atria or ventricles.

For the purposes of measuring mechanical restitution in accordance with the present invention, an implantable medical device system is equipped with at least one mechanical sensor of heart function. In the system shown in FIG. 1, right ventricular lead 16 is shown to include a mechanical sensor 30 which may be embodied as a pressure sensor, an accelerometer, a flow transducer, an acoustical sensor, or other sensor capable of generating a signal correlated to mechanical heart function. Sensor 30 is coupled to power supply circuitry and sensor signal processing circuitry contained in device 10 through lead conductors carried by lead 16. While a single mechanical sensor is shown positioned in the right ventricle for measuring mechanical heart function, it is recognized that one or more sensors of mechanical heart function may be positioned in operative relation to one or more heart chambers for measuring mechanical restitution properties or a correlate thereof. Furthermore, while mechanical sensor 30 is shown to be included on the same lead as pace/sense electrodes, a mechanical sensor may alternatively be provided on a separate lead body, which may be a transvenous, epicardial, subcutaneous or submuscular lead, or may be located on or within housing 11 of device 10 for receiving mechanical heart signals.

Alternative sensors for monitoring mechanical heart function could be embodied as impedance measuring electrodes. Impedance-based measurements of hemodynamic parameters such as stroke volume are known in the art as described, for example, in U.S. Pat. No. 5,578,064 issued to Prutchi.

While a particular multi-chamber device and lead system is illustrated in FIG. 1, methodologies included in the present invention may be adapted for use with other single chamber, dual chamber, or multichamber devices that are capable of sensing and processing cardiac electrical signals, sensing and processing cardiac mechanical signals, and delivering electrical stimulation pulses at controlled time intervals relative to an intrinsic or paced heart rate. As will be described below, electrical stimulation pulses will be injected following an intrinsic or paced primary systolic event to induce an extra systole at a known interval for the purposes of measuring a mechanical restitution parameter or surrogate thereof. Such devices will typically include at least electrical stimulation arrhythmia therapies, e.g., anti-tachycardia pacing therapies and cardioversion/defibrillation shock delivery, and may optionally include other electrical stimulation therapy delivery capabilities such as bradycardia pacing, cardiac resynchronization therapy, and extra systolic stimulation therapy.

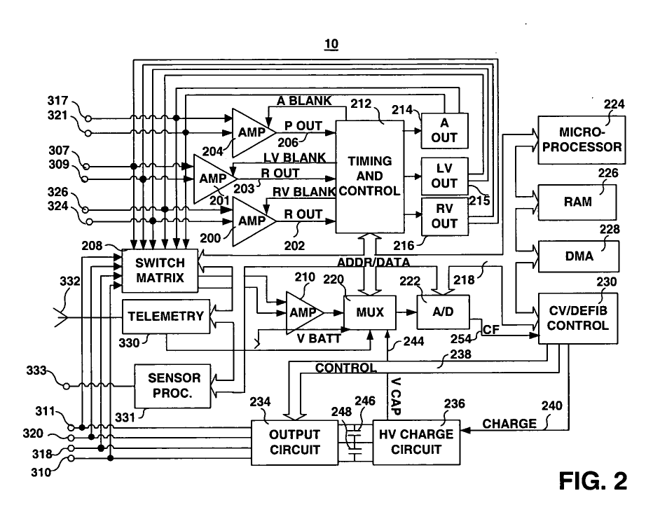

A functional schematic diagram of the device 10 is shown in FIG. 2. This diagram should be taken as exemplary of the type of device in which the invention may be embodied and not as limiting. The disclosed embodiment shown in FIG. 2 is a microprocessor-controlled device, but the methods of the present invention may also be practiced in other types of devices such as those employing dedicated analog or digital circuitry.

With regard to the electrode system illustrated in FIG. 1, the device 10 is provided with a number of connection terminals for achieving electrical connection to the leads 6, 15, and 16 and their respective electrodes. The connection terminal 311 provides electrical connection to the housing 11 for use as the indifferent electrode during unipolar stimulation or sensing. The connection terminals 320, 310, and 318 provide electrical connection to coil electrodes 20, 8 and 23 respectively. Each of these connection terminals 311, 320, 310, and 318 are coupled to the high voltage output circuit 234 to facilitate the delivery of high energy shocking pulses to the heart using one or more of the coil electrodes 8, 20, and 23 and optionally the housing 11. Connection terminals 311, 320, 310 and 318 are further connected to switch matrix 208 such that the housing 11 and respective coil electrodes 20, 8, and 23 may be selected in desired configurations for various sensing and stimulation functions of device 10.

The connection terminals 317 and 321 provide electrical connection to the tip electrode 17 and the ring electrode 21 positioned in the right atrium. The connection terminals 317 and 321 are further coupled to an atrial sense amplifier 204 for sensing atrial signals such as P-waves. The connection terminals 326 and 324 provide electrical connection to the tip electrode 26 and the ring electrode 24 positioned in the right ventricle. The connection terminals 307 and 309 provide electrical connection to tip electrode 9 and ring electrode 7 positioned in the coronary sinus. The connection terminals 326 and 324 are further coupled to a right ventricular (RV) sense amplifier 200, and connection terminals 307 and 309 are further coupled to a left ventricular (LV) sense amplifier 201 for sensing right and left ventricular signals, respectively.

The atrial sense amplifier 204 and the RV and LV sense amplifiers 200 and 201 preferably take the form of automatic gain controlled amplifiers with adjustable sensing thresholds. The general operation of RV and LV sense amplifiers 200 and 201 and atrial sense amplifier 204 may correspond to that disclosed in U.S. Pat. No. 5,117,824, by Keimel, et al., incorporated herein by reference in its entirety. Generally, whenever a signal received by atrial sense amplifier 204 exceeds an atrial sensing threshold, a signal is generated on output signal line 206. P-waves are typically sensed based on a P-wave sensing threshold for use in detecting an atrial rate. Whenever a signal received by RV sense amplifier 200 or LV sense amplifier 201 that exceeds an RV or LV sensing threshold, respectively, a signal is generated on the corresponding output signal line 202 or 203. R-waves are typically sensed based on an R-wave sensing threshold for use in detecting a ventricular rate.

Switch matrix 208 is used to select which of the available electrodes are coupled to a wide band amplifier 210 for use in digital signal analysis. Selection of the electrodes is controlled by the microprocessor 224 via data/address bus 218. The selected electrode configuration may be varied as desired for the various sensing, pacing, cardioversion and defibrillation functions of device 10. Signals from the electrodes selected for coupling to bandpass amplifier 210 are provided to multiplexer 220, and thereafter converted to multi-bit digital signals by A/D converter 222, for storage in random access memory 226 under control of direct memory access circuit 228. Microprocessor 224 may employ digital signal analysis techniques to characterize the digitized signals stored in random access memory 226 to recognize and classify the patient's heart rhythm employing any of the numerous signal processing methodologies known in the art.

The telemetry circuit 330 receives downlink telemetry from and sends uplink telemetry to an external programmer, as is conventional in implantable programmable medical devices, by means of an antenna 332. Data to be uplinked to the programmer and control signals for the telemetry circuit are provided by microprocessor 224 via address/data bus 218. Received telemetry is provided to microprocessor 224 via multiplexer 220. Numerous types of telemetry systems known for use in implantable devices may be used.

The remainder of the circuitry illustrated in FIG. 2 is an exemplary embodiment of circuitry dedicated to providing cardiac pacing, cardioversion and defibrillation therapies. The timing and control circuitry 212 includes programmable digital counters which control the basic time intervals associated with various single, dual or multi-chamber pacing modes, or anti-tachycardia pacing therapies delivered in the atria or ventricles. Timing and control circuitry 212 also determines the amplitude of the cardiac stimulation pulses under the control of microprocessor 224.

During pacing, escape interval counters within timing and control circuitry 212 are reset upon sensing of RV R-waves, LV R-waves or atrial P-waves as indicated by signals on lines 202, 203 and 206, respectively. In accordance with the selected mode of pacing, pacing pulses are generated by atrial output circuit 214, right ventricular output circuit 216, and left ventricular output circuit 215. The escape interval counters are reset upon generation of pacing pulses, and thereby control the basic timing of cardiac pacing functions, which may include bradycardia pacing, cardiac resynchronization therapy, and anti-tachycardia pacing.

The durations of the escape intervals are determined by microprocessor 224 via data/address bus 218. The value of the count present in the escape interval counters when reset by sensed R-waves or P-waves can be used to measure R—R intervals and P—P intervals for detecting the occurrence of a variety of arrhythmias.

In accordance with the present invention, timing and control 212 further controls the delivery of extra systolic stimulation pulses at selected extra systolic intervals (ESIs) following either sensed intrinsic systoles or pacing evoked systoles for the purposes of measuring a mechanical restitution parameter. The output circuits 214, 215 and 216 are coupled to the desired stimulation electrodes for delivering cardiac pacing therapies and extra systolic stimulation pulses via switch matrix 208.

The microprocessor 224 includes associated ROM in which stored programs controlling the operation of the microprocessor 224 reside. A portion of the memory 226 may be configured as a number of recirculating buffers capable of holding a series of measured R—R or P—P intervals for analysis by the microprocessor 224 for predicting or diagnosing an arrhythmia.

In response to the detection of tachycardia, anti-tachycardia pacing therapy can be delivered by loading a regimen from microcontroller 224 into the timing and control circuitry 212 according to the type of tachycardia detected. In the event that higher voltage cardioversion or defibrillation pulses are required, microprocessor 224 activates the cardioversion and defibrillation control circuitry 230 to initiate charging of the high voltage capacitors 246 and 248 via charging circuit 236 under the control of high voltage charging control line 240. The voltage on the high voltage capacitors is monitored via a voltage capacitor (VCAP) line 244, which is passed through the multiplexer 220. When the voltage reaches a predetermined value set by microprocessor 224, a logic signal is generated on the capacitor full (CF) line 254, terminating charging. The defibrillation or cardioversion pulse is delivered to the heart under the control of the timing and control circuitry 212 by an output circuit 234 via a control bus 238. The output circuit 234 determines the electrodes used for delivering the cardioversion or defibrillation pulse and the pulse wave shape.

In ICDs, the particular arrhythmia therapies are typically programmed into the device ahead of time by the physician, and a menu of therapies is typically provided. For example, on initial detection of tachycardia, an anti-tachycardia pacing therapy may be selected. On sustained or redetection of tachycardia, a more aggressive anti-tachycardia pacing therapy may be scheduled. If repeated attempts at anti-tachycardia pacing therapies fail, a higher-level cardioversion pulse therapy may be selected thereafter. The amplitude of a cardioversion or defibrillation shock may be incremented in response to failure of an initial shock or shocks to terminate fibrillation. Patents illustrating such pre-set therapy menus of anti-tachycardia therapies include U.S. Pat. No. 4,726,380 issued to Vollmann et al., U.S. Pat. No. 4,587,970 issued to Holley et al., and U.S. Pat. No. 4,830,006 issued to Haluska, incorporated herein by reference in their entirety. The use of such pre-programmed menus of arrhythmia therapies is anticipated to be benefited by the present invention in that the selection of initial therapies and the progression from less aggressive to more aggressive therapies may be influenced by the prediction of hemodynamic stability during a fast rate based on mechanical restitution measurements.

Device 10 is equipped with sensor signal processing circuitry 331 coupled to a terminal 333 for receiving a sensor signal from mechanical sensor 30. Sensor signal data, which may be digitized by A/D converter 222, is transferred to microprocessor 224 via data/address bus 218 such that a parameter of mechanical restitution may be determined according to algorithms stored in RAM 226. Sensors and methods for determining a mechanical restitution parameter as implemented in the previously-cited '408 patent to Mulligan may also be used in conjunction with the present invention. Methods described herein for measuring mechanical restitution may be implemented in software stored in RAM 226 executed by microprocessor 224. Alternatively, some or all operations for measuring a mechanical restitution parameter or surrogate thereof may be implemented in dedicated circuitry.

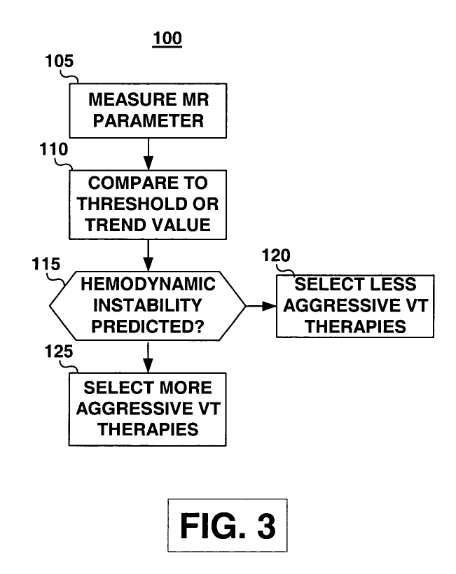

FIG. 3 is a flow chart summarizing steps included in a general method for practicing the present invention. At step 105, a mechanical restitution (MR) parameter is measured. As will be described in greater detail below, a mechanical restitution parameter may be a slope, time constant, or other characteristic of the steep portion (not the plateau portion) of a mechanical restitution curve. The MR parameter is compared at step 110 to a threshold value or range of values for determining if the parameter indicates a reduced ability to compensate hemodynamically for a rapid heart rate. A mechanical restitution trend may be determined from repeated MR parameter measurements such that a new measurement may be compared to the trend to determine if a worsening of the mechanical response to an extra systole is indicated.

If the MR parameter measured at step 105 predicts hemodynamic instability during a rapid heart rhythm, according to decision step 115 and based on comparison criteria used in comparison step 110, a clinician may use this information in programming a more aggressive menu of tiered VT therapies at step 125. If the MR parameter measured at step 105 does not predict hemodynamic instability as determined at decision step 115, the clinician may program VT therapies according to a nominal or generally less aggressive menu of tiered therapies. Selection of VT therapies at steps 120 and 125 based on the prediction made at step 115 may alternatively be made automatically be the implanted device. The implanted device may automatically select a more aggressive therapy menu if hemodynamic instability is predicted and a less aggressive therapy menu if hemodynamic stability is predicted.

The method 100 makes reference to programming arrhythmia therapies for treating VT in steps 120 and 125 since the ventricular contribution is more important than the atrial contribution to hemodynamic output and therefore is a greater determinant of hemodynamic stability. In this example, the MR parameter measured at step 105 will typically be based on a pressure, wall motion, or other sensor signal obtained from a mechanical sensor positioned for sensing ventricular activity. The methods described herein are expected to provide the greatest benefit when applied in the ventricular chamber, however the methods described herein may be adapted for use in an atrial chamber.

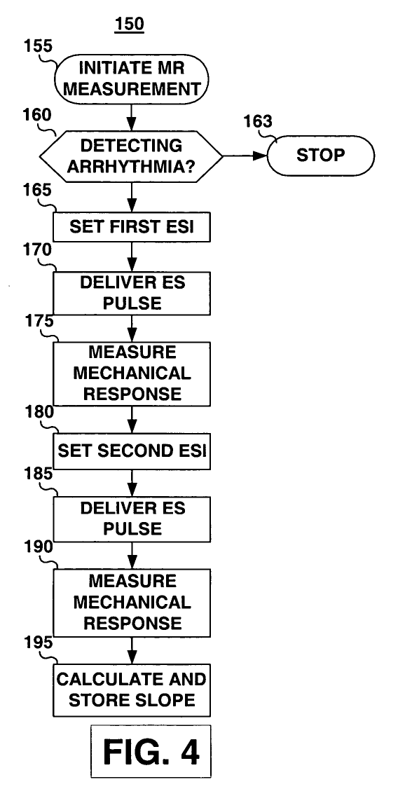

FIG. 4 is a flow chart summarizing steps included in a method for monitoring mechanical restitution for the purposes of predicting hemodynamic stability during a rapid heart rhythm. At step 155, a mechanical restitution measurement is initiated. MR measurements may be made on a scheduled, periodic basis, e.g., daily, weekly, or monthly. MR measurements may also be initiated manually be a clinician using an external programming device. At decision step 160, method 150 verifies that the implantable device is not currently detecting an arrhythmia. Preferably, the MR measurement is performed during a normal sinus rhythm such that injection of an extra systolic stimulation pulse for measuring a MR parameter does not interfere with an already accelerated or unstable rhythm. If an arrhythmia episode is ongoing, method 150 is terminated at step 163 and no MR measurement is made at this time.

As long as the device is not currently detecting an arrhythmia, method 150 proceeds to step 165 and sets a first extra systolic interval (ESI). An extra-systolic (ES) pulse is delivered at step 170 following a sensed or paced primary systole and the ESI. The mechanical response to the extra systole is measured at step 175. The mechanical response is measured using a mechanical sensor as described previously that provides a signal correlated to mechanical or hemodynamic heart function. As such, the mechanical response measured at step 175 may be a peak blood pressure, a maximum rate of rise in blood pressure (dP/dt), maximum wall acceleration, impedance based stroke volume, or the like.

At step 180, a second ESI is set and an ES pulse is delivered at step 185 following a paced or sensed primary systole and the second ESI. The mechanical response to the extra systole at the second ESI is measured at step 190. One or more ES pulses may be delivered at each ESI with the mechanical responses measured at steps 175 and 190 performed for each ES pulse and averaged for a given ESI.

At step 195, the slope of the MR curve between the two ESI applied is calculated and stored. The slope of the MR curve may be estimated based on only two points defined by the two ESIs applied in method 150 and the corresponding mechanical response measurements. However, two or more ESIs may be applied to obtain points on the MR curve and calculate a corresponding slope. The ESIs applied are preferably selected such that the points fall on the steep portion of the mechanical restitution curve.

The MR curve slope determined at step 195 may then be used by method 100 of FIG. 3 for comparison to a threshold or trend value. A relatively low slope indicated a blunted hemodynamic response to the ES and is a predictor of hemodynamic instability during a rapid rhythm.

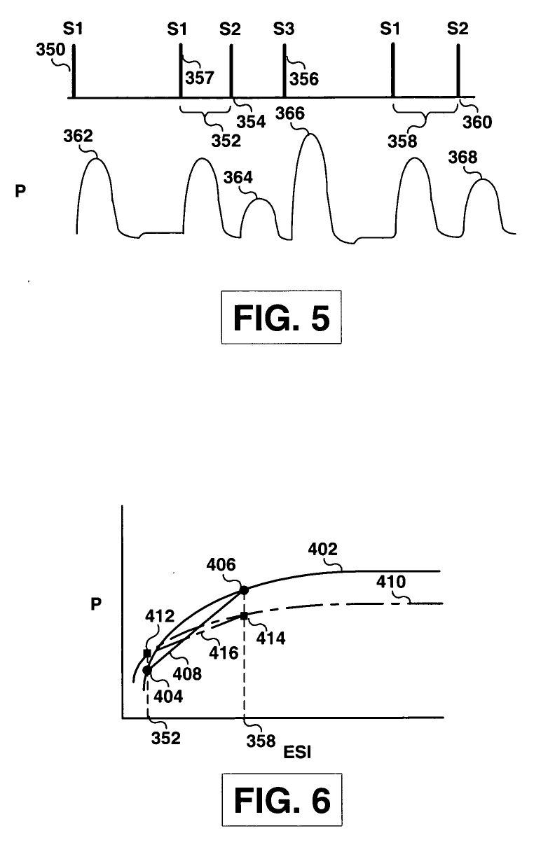

FIG. 5 is a time line depicting the application of ES pulses and the mechanical response to the extra systole which may be measured for estimating a slope of the MR curve. The S1 pulse 350 represents a primary systolic event, which may be a paced or sensed event. The mechanical response is represented by a ventricular pressure (P) signal. Each S1 350 event is accompanied by a “normal” pulse pressure response having a peak pressure 362. Following an S1 pulse, a first ESI 352 is applied after which an ES pulse (S2) 354 is delivered. The amplitude of the mechanical response to the ES pulse is reduced, resulting in a considerably lower peak pressure 364. The post-extra systolic event (S3) 356 following the ES pulse (S2) 354 is typically associated with an enhanced mechanical response as indicated by the increased peak pressure 366 compared to the primary systolic peak pressure 362.

Following a subsequent primary systole (S1) 357 event, a second ESI 358 is applied which is longer than the first ESI 352. The ES pulse (S2) 360 produces a mechanical response that is relatively higher than the response to the extra systole (S2) 354 at the shorter ESI 352 but reduced compared to the “normal” mechanical response to a primary systole (S1) 357. Thus, the peak pressure 368 measured following the second, longer ESI 358 is greater than the peak pressure 364 measured following the first, shorter ESI 352 but still less than the primary systolic peak pressure 362. Using the peak pressures 364 and 368 measured at two different ESIs 352 and 358, respectively, the slope of a portion of the MR curve may be calculated.

FIG. 6 is an illustration of mechanical restitution curves representing a normal, healthy hemodynamic response and an abnormal, reduced hemodynamic response to extra systoles occurring over a range of ESIs. ESI is plotted along the X-axis and a measure of mechanical or hemodynamic heart function is plotted along the Y-axis. In this example, ventricular pulse pressure (P) is plotted along the Y-axis. MR curve 402 represents a typical MR curve for a normal, healthy person. A steep phase is followed by a plateau phase. The steep phase represents the increasing mechanical response to extra systoles occurring between a very short ESI, which results in no mechanical response, to the shortest ESI that produces a maximum mechanical response. As ESI is increased further, the mechanical response does not increase producing the plateau phase of the MR curve 402.

By measuring the mechanical response at two ESIs along the steep phase of the MR curve, a slope of the steep phase can be calculated. With reference to the first, shorter ESI 352 as shown in FIG. 5, a first peak pressure measurement is made along MR curve 402 and plotted as point 404. A second peak pressure measurement is made during an extra systole following the second, longer ESI 358 and plotted as point 406. The slope 408 between points 404 and 406 represents a MR parameter that may be used as a metric of the mechanical response to an extra systole. The relatively steep slope 408 indicates that the patient is likely to be able to respond favorably to a rapid rhythm by quickly compensating hemodynamically for the fast heart rate. In such a patient, a detected VT may be predicted to be a stable VT based on the steep slope of the MR curve. It may be desirable to initially attempt to terminate a VT predicted to be hemodynamically stable with less aggressive anti-tachycardia pacing.

MR curve 410 represents the mechanical response to extra systoles in an unhealthy patient. In the same manner as described above, points 412 and 414 may be determined by measuring the mechanical response during an extra systole delivered at the first, shorter ESI 352 and the second, longer ESI 358. The calculated slope 416 is lower than the slope 408 in a healthy person reflecting the relatively flatter steep phase of the MR curve 410 compared to the steep phase of the MR curve 402 in a healthy person. The hemodynamic response to an extra systole in an unhealthy person is blunted due to impaired calcium handling. This blunted hemodynamic response suggests that such a patient will be unlikely to tolerate a rapid rhythm. A detected VT is in such a patient is likely to be unstable with insufficient hemodynamic output. A more aggressive approach to treating VT may therefore be desirable. Thus, by monitoring a MR parameter, stable and unstable VT may be predicted and used in selecting pre-programmed VT therapies.

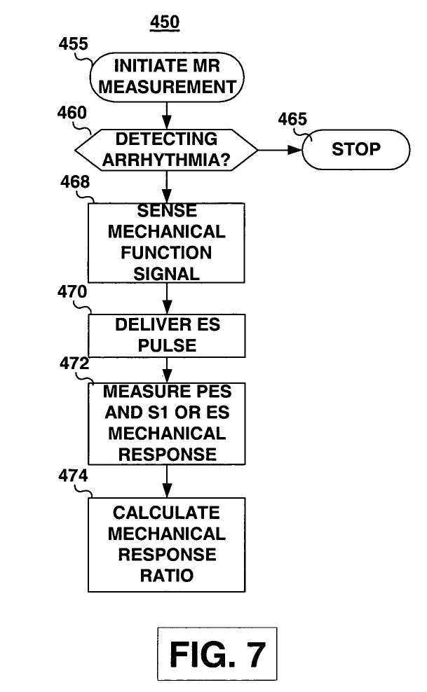

FIG. 7 is a flow chart summarizing steps included in an alternative method for predicting the hemodynamic response to a fast rhythm. Heart failure patients are suspected to have a reduced force/frequency response. A normal mechanical response to an extra systole produces enhanced mechanical function on post-extra systolic beats, as illustrated in FIG. 5. The pulse pressure measured on a post-extra systolic beat is therefore expected to be greater than the pulse pressure measured on the primary systole, preceding the extra systole, and much greater than the pulse pressure on the extra systole. In patients expected to have an unstable hemodynamic response to a fast rate, the mechanical response on post-extra systolic beats is expected to be reduced. This damped or reduced post-extra systolic mechanical response may be explained in part by the inability of the heart to lengthen its diastolic interval to accommodate shortened systolic intervals. In a healthy heart, the diastolic interval will dynamically change to accommodate changing systolic intervals. As such, measurement of the post-extra systolic mechanical function may be used as an alternative or in addition to the measurement of the mechanical function on the extra systole for predicting hemodynamic stability during a rapid ventricular rate.

At step 455, a MR measurement is initiated as described previously. Method 450 verifies that the device is not currently detecting an arrhythmia at decision step 460 to avoid delivering an extra systolic pulse during an accelerated rhythm. If an arrhythmia episode is being detected, method 450 is terminated at step 465.

If an arrhythmia is not being detected, the mechanical function signal is sensed at step 468 to allow measurement of the mechanical function, on the post-extra systolic beat and at least one or both of the extra systolic beat and the preceding primary systolic beat as will be described below. An extra systolic pulse is delivered at step 470 at a predetermined ESI. At step 472, the mechanical response on the first post-extra systolic beat (S3 as shown in FIG. 5) is measured. Additionally, the mechanical response to either or both the primary systolic beat (S1 as shown in FIG. 5) and the extra systolic beat (S2 as shown in FIG. 5) are measured.

A mechanical response ratio may then be calculated at step 474 as the ratio of the mechanical function on the first post extra systole (PES) to the primary systole (S1) and/or the ratio of the mechanical function on the first PES to extra systole (ES). In a patient having a stable response to a fast rhythm, the PES mechanical function is expected to be greater than the S1 mechanical function and much greater than the ES mechanical function.

The mechanical response ratio calculated at step 474 may thus be used as a substitute MR parameter in method 100 of FIG. 3 for predicting the hemodynamic response to a fast rhythm. The ratio may be compared to a threshold or trend value at step 110 of FIG. 3 wherein if the ratio is less than an expected value, indicating a blunted post-extra systolic mechanical response, hemodynamic instability may be predicted at step 115 of method 100.

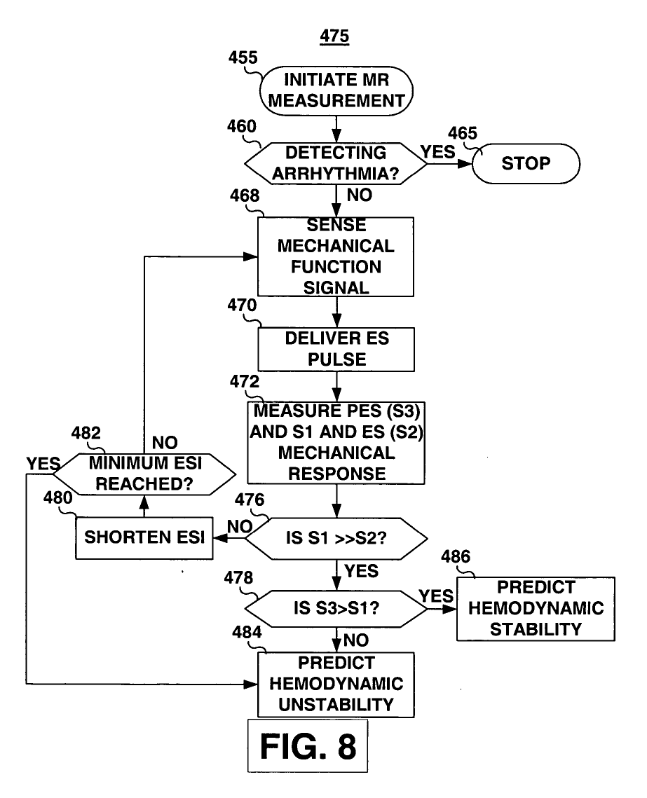

FIG. 8 is a flow chart providing details of an alternative method for predicting the hemodynamic response to a fast rhythm by using the PES mechanical function measurement. Steps 455 through 472 of method 475 correspond to identically-labeled steps included in method 450 of FIG. 7 except that at step 472 the mechanical function is measured on all of the PES or S3 beat, the primary systole or S1 beat, and the ES or S2 beat. After obtaining these measurements, the mechanical function on the primary systole or S1 beat is compared to the mechanical response on the ES or S2 beat at decision step 476. If the S1 beat is considerably greater than the S2 beat, method 475 proceeds to step 478 to evaluate the mechanical response of the PES or S3 beat.

However, if the S1 beat is not considerably greater than the S2 beat, as determined at decision step 476, the ESI may have been too long to achieve the expected PES effect or the patient may have an abnormal response to changing rates. If the ESI is too long, the S2 beat will be very similar to the S1 beat and, likewise, the S3 beat will be very similar to the S1 and S2 beats. Therefore, the ESI used to inject an ES pulse may be shortened at step 480 up to some minimum ESI as determined at decision step 482. The ESI is preferably not shortened to the point that the ES pulse is delivered during the so-called vulnerable period, which may induce arrhythmias in some patients. If the minimum ESI has not been reached, method 475 may return to step 468 to continue sensing the mechanical function signal and proceed with delivering a new ES pulse at the shortened ESI.

If a minimum ESI is reached without substantial weakening of the mechanical function on the ES (S2) beat relative to the primary S1 beat, method 475 concludes at step 484 with the prediction of hemodynamic instability in response to a rapid rhythm. The absence of mechanical weakening on the S2 beat relative to the S1 beat may be evidence of abnormal mechanical restitution. In light of such abnormal mechanical function, an unstable hemodynamic response to a rapid rhythm is expected.

If the ES mechanical function is found to be substantially weakened compared to the primary S1 beat, as determined at decision step 476, method 475 proceeds to step 478 to compare the mechanical function on the post-extra systolic S3 beat to the primary S1 beat. If the post-extra systolic mechanical function is enhanced compared to the primary systole (i.e., the ratio of the S1/S3 mechanical function is relatively low), a stable hemodynamic response to a rapid rhythm is predicted at step 486. If the post-extra systolic mechanical function is not greatly enhanced (i.e., the ratio of S1/S3 mechanical function is relatively high), an unstable hemodynamic response to a rapid rhythm is predicted at step 484. Alternatively, the ratio of S2/S3 mechanical function may be examined. A relatively low S2/S3 ratio predicts a stable response, and a relatively high S2/S3 ratio predicts an unstable response to a rapid rhythm. A blunted mechanical response on a PES is expected to be associated with a relatively flattened MR curve compared to a normal MR curve. Thus, evaluation of the post-extra systolic mechanical function (relative to the primary S1 or extra systolic S2 mechanical function) may be used as an alternative to determining a MR curve parameter for use in predicting a patient's hemodynamic response to a fast rate.

FIG. 9 is a graph depicting ventricular pulse pressure plotted against diastolic interval. As DI interval increases, the generated pulse pressure (PP) increases up to a maximum pulse pressure. Normally, as the cardiac cycle length shortens, the diastolic interval (DI) is lengthened. The pulse pressure generated as a result of the lengthened DI is enhanced as shown by the curve in FIG. 9. This response is referred to as the force-frequency response; at increased frequency, developed pressure is increased.

Some patients are unable to significantly lengthen their diastolic interval (DI) in response to a faster rate and therefore are unable to increase the pulse pressure generated on each beat. These patients may experience hemodynamic insufficiency during the fast rate due to the impaired force-frequency response. Patients that are able to lengthen their DI within the first few beats of a rapid rhythm will generally experience hemodynamic stability during the fast rate due to the enhanced pressure development resulting from the longer DI. Therefore, measurement of the DI during the first few beats of a rapid rhythm or in response to an extra systole may provide a surrogate parameter for predicting the hemodynamic response to a rapid rhythm.

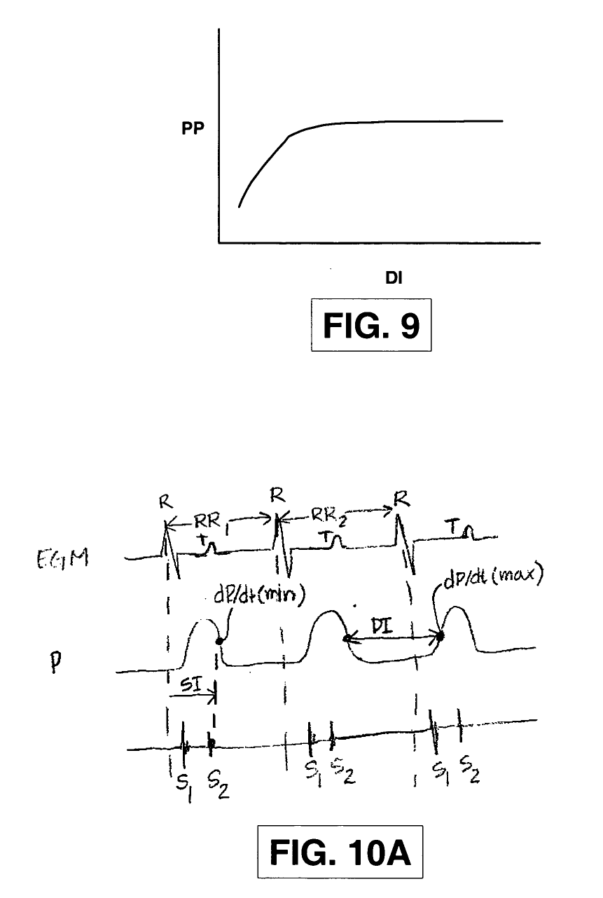

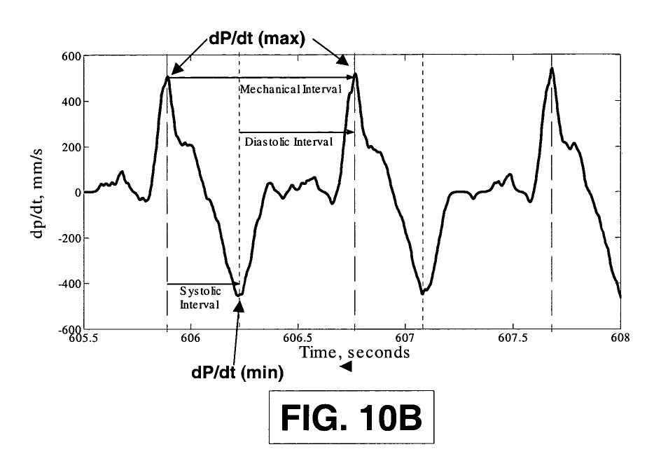

FIG. 10 is a depiction of an EGM signal, a pressure signal, and an accelerometer signal illustrating methods for measuring the DI. Two R—R intervals are depicted on the EGM signal, RR₁ and RR₂. The systolic interval (SI) may be measured as the time from a detected R-wave to the minimum derivative of the pressure signal, dP/dt(min), which corresponds approximately in time to the start of isovolumic relaxation and the closure of the aortic and pulmonic valves, which creates the second heart sound, S2, measured on the accelerometer signal. The diastolic interval on the subsequent cardiac cycle will depend on the previous SI and the current R—R interval. Thus, DI may be determined as the difference between the current R—R interval measured from the EGM signal and the previous SI measured between a sensed R-wave and the subsequent dP/dt(min) on a pressure signal or the second heart sound on an accelerometer signal. The second heart sound also corresponds approximately in time with the T-wave of an EGM signal. As such, DI could be estimated from the EGM signal by measuring the interval between a sensed T-wave and a subsequently sensed R-wave.

Alternatively, the diastolic interval may be estimated using only a mechanical signal of heart function. For example, the DI may be estimated as the interval between the second heart sound (aortic and pulmonic valve closing) and the subsequent first heart sound (atrioventricular valve closing), which may be determined from an accelerometer signal as shown in FIG. 10A. FIG. 10B is a depiction of a dP/dt curve illustrating an alternative method for measuring DI. The cardiac mechanical cycle extends between two consecutive dP/dt peaks (dP/dt max). The systolic interval begins at a dP/dt max and ends at a consecutive dP/dt min. The DI may be measured as the interval between dP/dt(min) and the subsequent dP/dt(max), as indicated, which corresponds to the opening of the aortic valve and the onset of rapid ejection. It is recognized that numerous methods may be conceived for measuring or estimating the DI based on electrical and/or mechanical signals of cardiac function.

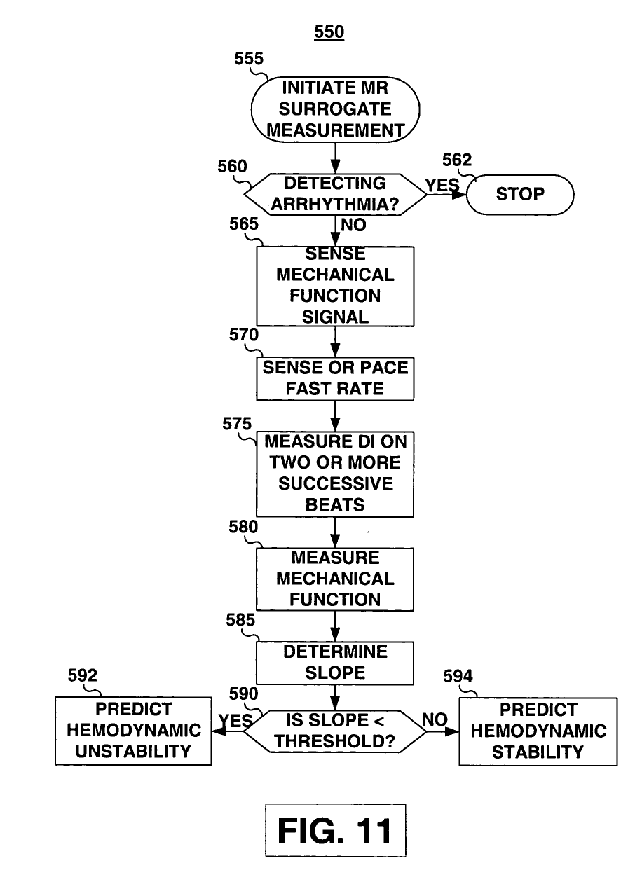

FIG. 11 is a flow chart summarizing steps included in an alternative method for predicting the hemodynamic response to a fast rate based on measurements of the DI. Measurements of the DI are used as a surrogate measure for an MR parameter in predicting the hemodynamic response to a fast rate. As such, at step 555, an MR surrogate measurement is initiated. In the same manner as described previously, this initiation step may be triggered to occur on a scheduled, periodic basis or by a clinician. Preferably, method 550 is performed during a stable rhythm therefore at decision step 560, verification is made that no arrhythmia is currently being detected. Otherwise, method 550 is terminated at step 562.

If no arrhythmia is currently being detected, sensing of a mechanical function signal is enabled at step 565. An object of method 550 is to determine if the patient is able to lengthen their DI and have a normal force-frequency response to a fast rate. Therefore at step 570, either a fast intrinsic rate is sensed or the heart may be paced at a fast rate. A fast intrinsic rate may be induced by asking the patient to exercise. At the onset of the fast intrinsic or paced rate, the DI is measured on at least two successive heart beats, which may be consecutive or separated by one or more beats. The DI measurements are preferably made within the first several beats after the onset of a fast rate. However, one or more DI measurements may additionally be made after the first several beats when the DI has presumably reached a steady-state. DI measurements may be made as described previously in conjunction with FIG. 10.

In addition to measuring the DI, the mechanical function signal is processed for obtaining a mechanical function measurement for the corresponding cardiac cycles as indicated at step 580. By measuring the mechanical function and the DI, a mechanical function versus DI curve may be approximated. By having at least two point on this curve based on two DI measurements and corresponding mechanical function parameters, a slope may be determined as indicated by step 585.

The slope may be compared to a threshold value at decision step 590 for determining if the slope is indicative of a normal force-frequency response. If a relatively low slope is measured, i.e., little increase in mechanical function with small or no change in DI, the patient is predicted to have an unstable hemodynamic response to a fast rate as indicated by step 592. If the slope is relatively high, the patient is expected to respond appropriately to a fast rate by increasing the DI and mechanical function during a fast rate. Hemodynamic stability during a fast rhythm is predicted at step 594.

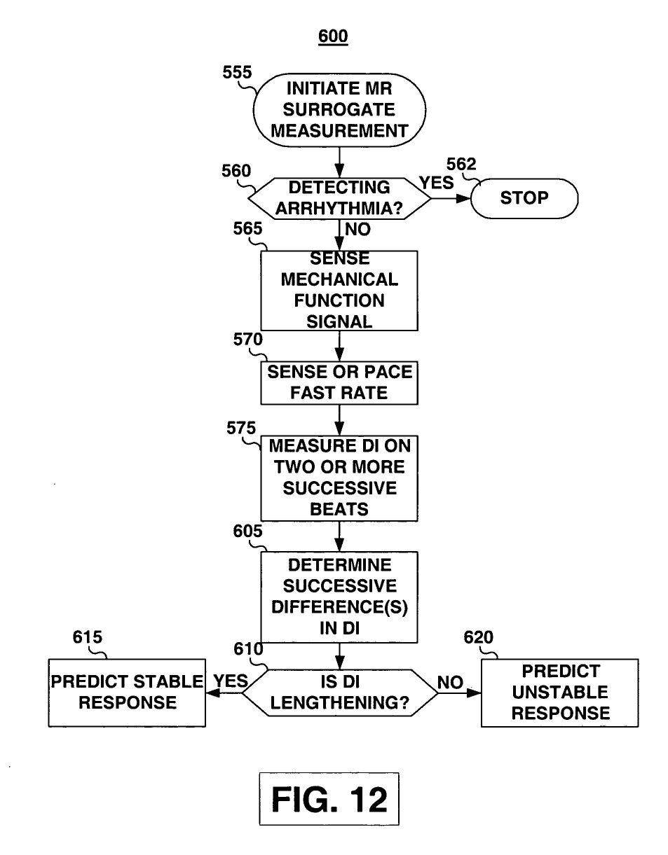

FIG. 12 is a flow chart summarizing steps included in an alternative method for using measurements of DI as a surrogate to determining a MR parameter in predicting the hemodynamic response to a fast rate. In method 600, steps 555 through 575 correspond to identically labeled steps included in method 550 of FIG. 11. At step 605, the successive differences between DIs measured at step 575 are determined.

If the successive DI differences indicate a pattern of lengthening DI, as determined at decision step 610, a stable hemodynamic response to a fast rhythm is predicted at step 615. If a pattern of lengthening DI is not indicated, an unstable hemodynamic response to a fast rhythm is predicted at step 620. Patients unable to extend their DI at the start of a fast rhythm will not be able to “move up” the steep phase of the MR curve and increase their hemodynamic response. Thus, by examining changes in DI alone at the onset of a fast rhythm, a prediction of the hemodynamic response to a fast rhythm may be made.

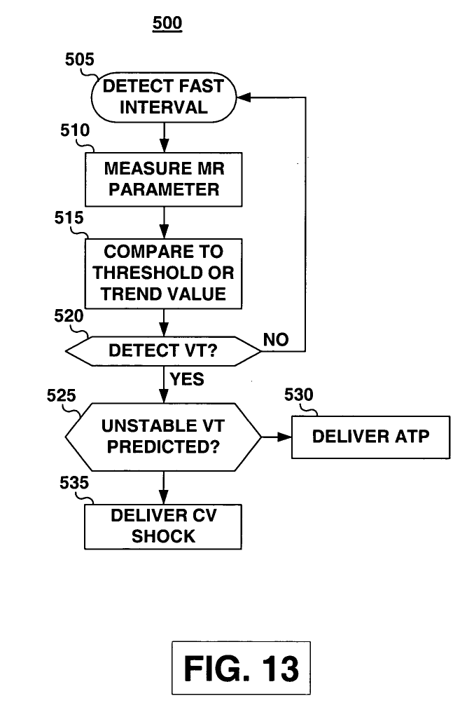

FIG. 13 is a flow chart summarizing steps included in a method for using MR parameter or surrogate measurements for discriminating stable from unstable VT during detection of a VT episode. Heretofore, methods described herein have been intended for use during a stable rhythm for predicting the response to a fast rhythm occurring in the future. However, MR parameters or surrogate parameters may be determined at the onset of a fast rhythm to predict if the patient is going to respond in a hemodynamically stable manner or if the patient is likely to experience hemodynamic insufficiency. Such discrimination between stable and unstable VT at the onset or just prior to a VT detection may be used by an implantable device to automatically select a VT therapy.

Method 500 is initiated at step 505 upon detecting a fast interval. For example, a fast R—R interval falling within a VT detection zone may be detected. At step 510 a MR parameter is measured. A MR parameter may be measured by measuring the mechanical response to the first fast interval detected and one or more of the succeeding intervals falling in the VT detection zone. Alternatively the mechanical response of the systole occurring just prior to the fast interval may be measured. By obtaining two or more measurements of the mechanical response to at least two different R—R intervals, a slope may be calculated representing an MR curve slope. As described above, surrogate parameters may alternatively or additionally be measured, such as the slope of a mechanical function versus DI curve or successive DI differences.

The parameter measured at step 510 may be compared to a threshold value or range of values or previously determined MR parameter trend. If VT detection criteria are met, as determined at decision step 520, method 500 proceeds to decision step 525. If VT detection criteria are not met, method 500 may return to step 505 to await detection of the next fast interval. VT detection criteria may be based on known arrhythmia detection algorithms including interval analysis, interval pattern analysis and/or EGM morphology analysis.

If VT is detected, method 500 determines if the VT is predicted to be hemodynamically unstable based on the comparison made at step 515. If unstable VT is predicted, an aggressive VT therapy may be selected as indicated at step 535. For example, a CV shock may be delivered immediately to quickly terminate the VT before the patient experiences symptoms associated with hemodynamic insufficiency. If the VT is predicted to be stable, a less aggressive VT therapy menu may be selected according to previous programming. For example, anti-tachycardia pacing (ATP) may be initiated as indicated at step 530.

Some of the techniques described above may be embodied as a computer-readable medium comprising instructions for a programmable processor such as microprocessor 224. The programmable processor may include one or more individual processors, which may act independently or in concert. A “computer-readable medium” includes but is not limited to any type of computer memory such as floppy disks, conventional hard disks, CR-ROMS, Flash ROMS, nonvolatile ROMS, RAM and a magnetic or optical storage medium. The medium may include instructions for causing a processor to perform any of the features described above for delivering therapy in an implantable medical device according to the present invention.

Thus, a system and associated methods have been described herein for use in predicting the hemodynamic response to fast rhythms. The predicted hemodynamic response may be used in selecting arrhythmia therapies to allow a more aggressive therapy approach to be taken when unstable rhythms are predicted. While numerous variations have been described in considerable detail, it is recognized that one of skill in the art, having the benefit of the teachings provided herein, may conceive of alternative approaches for measuring or estimating MR parameters or surrogate parameters that are useful in predicting the hemodynamic response to fast rhythms. The methods described herein are intended to be illustrative, not limiting, with regard to the following claims.

What is claimed is:

1. A method, comprising:
measuring a mechanical restitution parameter;
performing a comparative analysis of the measured mechanical restitution parameter; and
predicting the hemodynamic response to a fast heart rate based on the comparative analysis.

2. The method of claim 1 further comprising selecting an arrhythmia therapy based on the predicted hemodynamic response to a fast heart rate.

3. The method of claim 1 wherein measuring the mechanical restitution parameter comprises measuring a slope of a mechanical restitution curve.

4. The method of claim 3 wherein measuring the mechanical restitution curve slope further comprises:
delivering an extra systolic stimulation pulse; and
measuring a mechanical in response to the extra systolic stimulation pulse.

5. The method of claim 4 wherein delivering an extra systolic stimulation pulse includes delivering multiple extra systolic stimulation pulses delivered at at least two different extra systolic intervals.

6. The method of claim 1 wherein measuring the mechanical restitution parameter further comprises:
measuring a mechanical response on a primary systolic beat;
measuring a mechanical response on an extra systolic beat;
measuring a mechanical response on a post-extra systolic beat.

7. The method of claim 6 wherein performing the comparative analysis includes comparing the mechanical response of the post-extra systolic beat relative to the mechanical response of the primary systolic beat or the mechanical response of the extra systolic beat.

8. The method of claim 1 wherein measuring a mechanical restitution parameter further comprises:
measuring a diastolic interval;
measuring a mechanical response to two or more measured diastolic intervals; and
determining a slope of the curve defined by the measured mechanical responses at the two or more measured diastolic intervals.

9. The method of claim 1 wherein measuring a mechanical restitution parameter further comprises:
measuring consecutive diastolic intervals during the onset of a rapid heart rate; and
determining successive differences of the measured consecutive diastolic intervals.

10. A method of delivering a therapy to a patient from a medical device, comprising:
determining a parameter associated with the mechanical restitution of the patient;
determining whether the parameter indicates a reduction in hemodynamic compensation responsive to an increased heart rate; and
adjusting the therapy delivery in response to the determining whether the parameter indicates a reduction in hemodynamic compensation.

11. The method of claim 10, wherein determining a parameter comprises:
setting a first extra systolic interval;
measuring a first mechanical response to a first pulse delivered following one of a sensed and a paced primary systole and the first extra systolic interval;
setting a second extra systolic interval; and
measuring a second mechanical response to a second pulse delivered following one of a sensed and a paced primary systole and the second extra systolic interval.

12. The method of claim 11, wherein determining whether the parameter indicates a reduction in hemodynamic compensation comprises determining a slope corresponding to the first mechanical response and the second mechanical response, wherein indication of a reduction increases as the determined slope decreases.

13. The method of claim 11, wherein the second extra systolic interval is greater than the first extra systolic interval.

14. The method of claim 11, wherein the first extra systolic interval and the second extra systolic interval correspond to a sloped portion of a mechanical restitution curve having a slope greater than a slope corresponding to other than the sloped portion.

15. The method of claim 11, wherein determining whether the parameter indicates a reduction in hemodynamic compensation comprises determining whether the second mechanical response is greater than the first mechanical response.

16. The method of claim 11, further comprising:
measuring a third mechanical response to a third pulse delivered at a predetermined interval subsequent to the first pulse; and
determining a first ratio of the third mechanical response to the first mechanical response and a second ration of the third mechanical response to the second mechanical response, and wherein a reduction in hemodynamic compensation is determined in response to the first ratio and the second ratio.

17. The method of claim 10, wherein the parameter corresponds to a slope of a mechanical function and a diastolic interval curve.

18. The method of claim 10, wherein the parameter corresponds to successive differences between diastolic interval measurements.

19. An apparatus for delivering a therapy to a patient, comprising
means for determining a parameter associated with the mechanical restitution of the patient;
means for determining whether the parameter indicates a reduction in hemodynamic compensation responsive to an increased heart rate; and
means for adjusting the therapy delivery in response to the determining whether the parameter indicates a reduction in hemodynamic compensation.

20. The apparatus of claim 19, wherein means for determining a parameter comprises:
means for setting a first extra systolic interval;
means for measuring a first mechanical response to a first pulse delivered following one of a sensed and a paced primary systole and the first extra systolic interval;
means for setting a second extra systolic interval; and
means for measuring a second mechanical response to a second pulse delivered following one of a sensed and a paced primary systole and the second extra systolic interval.

21. The apparatus of claim 20, wherein means for determining whether the parameter indicates a reduction in hemodynamic compensation comprises means for determining a slope corresponding to the first mechanical response and the second mechanical response, wherein indication of a reduction increases as the determined slope decreases.

22. The apparatus of claim 20, wherein the second extra systolic interval is greater than the first extra systolic interval.

23. The apparatus of claim 20, wherein the first extra systolic interval and the second extra systolic interval correspond to a sloped portion of a mechanical restitution curve having a slope greater than a slope corresponding to other than the sloped portion.

24. The apparatus of claim 20, wherein means for determining whether the parameter indicates a reduction in hemodynamic compensation comprises means for determining whether the second mechanical response is greater than the first mechanical response.

25. The apparatus of claim 20, further comprising:
means for measuring a third mechanical response to a third pulse delivered at a predetermined interval subsequent to the first pulse; and
means for determining a first ratio of the third mechanical response to the first mechanical response and a second ration of the third mechanical response to the second mechanical response, and wherein a reduction in hemodynamic compensation is determined in response to the first ratio and the second ratio.

26. The apparatus of claim 19, wherein the parameter corresponds to a slope of a mechanical function and a diastolic interval curve.

27. The apparatus of claim 19, wherein the parameter corresponds to successive differences between diastolic interval measurements.

28. A computer readable medium having computer executable instructions for performing a method comprising:
means for determining a parameter associated with the mechanical restitution of the patient;
means for determining whether the parameter indicates a reduction in hemodynamic compensation responsive to an increased heart rate; and
means for adjusting the therapy delivery in response to the determining whether the parameter indicates a reduction in hemodynamic compensation.
